(19) United States
(12) Patent Application Publication
(10) Pub. No.: US 2022/0282208 A1
(43) Pub. Date: Sep. 8, 2022

(54) METHODS OF ISOLATING T CELL POPULATIONS

(71) Applicants: Douglas C. PALMER, North Bethesda, MD (US); Anna PASETTO, Stockholm (SE); Nicholas P. RESTIFO, Chevy Chase, MD (US); Steven A. ROSENBERG, Potomac, MD (US); THE UNITED STATES OF AMERICA,AS REPRESENTED BY THE SECRETARY,DEPARTMENT OF HEALTH AND HUMAN SERVICES, Bethesda, MD (US)

(72) Inventors: Douglas C. Palmer, North Bethesda, MD (US); Anna Pasetto, Stockholm (SE); Nicholas P. Restifo, Chevy Chase, MD (US); Steven A. Rosenberg, Potomac, MD (US)

(73) Assignee: THE UNITED STATES OF AMERICA,AS REPRESENTED BY THE SECRETARY,DEPARTMENT OF HEALTH AND HUMAN SERVICES, Bethesda, MD (US)

(21) Appl. No.: 17/636,206

(22) PCT Filed: Sep. 18, 2020
(86) PCT No.: PCT/US2020/051548

§ 371 Date: Feb. 17, 2022

Related U.S. Application Data
(60) Provisional application No. 62/902,184, filed on Sep. 18, 2019.

(51) Int. Cl. C12N 5/00; G01N 33/58; G01N 33/50; C12N 5/078; A61K 35/17; C07K 14/725

(52) U.S. Cl. CPC C12N 5/0093; C12N 5/0087; G01N 33/582; G01N 33/5094; C12N 5/0634; A61K 35/17; C07K 14/7051; C12N 2501/2302; C12N 2501/2307; C12N 2501/2312; C12N 2501/2315

(57) ABSTRACT

Provided are methods of producing an isolated population of cells for adoptive cell therapy comprising use of at least one cell permeable Ca²⁺ dye. Further embodiments of the invention provide isolated populations of cells produced by the methods, related pharmaceutical compositions, and related methods of treating or preventing cancer in a patient.

### CROSS-REFERENCE TO RELATED APPLICATION

This patent application claims the benefit of co-pending U.S. Provisional Patent Application No. 62/902,184, filed Sep. 18, 2019, which is incorporated by reference in its entirety herein.

### STATEMENT REGARDING FEDERALLY SPONSORED RESEARCH OR DEVELOPMENT

This invention was made with Government support under project number Z01ZIA BC010763 by the National Institutes of Health, National Cancer Institute. The Government has certain rights in this invention.

### BACKGROUND OF THE INVENTION

Adoptive cell therapy (ACT) using cancer-reactive T cells can produce positive clinical responses in some cancer patients. Nevertheless, several obstacles to the successful use of ACT for the treatment of cancer and other conditions remain. For example, the current methods used to produce cancer-reactive T cells require significant time and may not readily identify the desired T cell receptors that bind cancer targets. Accordingly, there is a need for improved methods of obtaining an isolated population of cells for ACT.

### BRIEF SUMMARY OF THE INVENTION

An embodiment of the invention provides a method of producing an isolated population of cells for adoptive cell therapy, the method comprising: a) providing a tumor sample containing T cells and tumor cells from a patient having a tumor; b) separating the T cells from the tumor cells of the tumor sample of a) to produce a separated population of T cells and a separated population of tumor cells; c) exposing the separated population of T cells of b) to at least one non-cytotoxic cell permeable Ca²⁺ dye to produce dyed T cells; d) exposing target cells to at least one non-cytotoxic cell membrane dye to produce dyed target cells, wherein the target cells are the separated population of tumor cells of b) or antigen presenting cells (APCs), wherein the separated population of tumor cells of b) express one or more tumor antigens and the APCs are loaded with or express one or more tumor antigens; e) exposing the dyed T cells to the dyed target cells under conditions sufficient for at least a portion of the dyed T cells to specifically bind to the one or more tumor antigens of the dyed target cells; f) identifying the dyed T cells which exhibit both (i) specific binding to the dyed target cells and (ii) absorption of a level of the at least one cell permeable Ca²⁺ dye sufficient to indicate T cell receptor activation; g) separating the dyed T cells identified to exhibit both (i) and (ii) from dyed T cells which fail to exhibit both (i) and (ii); h) obtaining a sequence of a T cell receptor from a T cell which exhibits both (i) and (ii); and i) inserting the sequence of the T cell receptor of h) into a peripheral blood mononuclear cell (PBMC) to provide an isolated population of cells for adoptive cell therapy.

Further embodiments of the invention provide isolated populations of cells produced by the method, related pharmaceutical compositions, and related methods of treating or preventing cancer in a patient.

### BRIEF DESCRIPTION OF THE SEVERAL VIEWS OF THE DRAWING(S)

FIGS. 1A-1B present representative fluorescence-activated cell sorting (FACS) data for human TIL (FIG. 1A) and melanoma tumor cells (FIG. 1B) that were stained with a violet cell tracker dye and an APC cell tracker dye. The numbers in the quadrants represent the number of cells in the circled area of the plot.

FIGS. 2A-2D present FACS data for melanoma tumor cells that were stained with a violet cell tracker dye (surface stain) and an APC cell tracker dye (cell permeable calcium dye). The numbers in the quadrants represent the number of cells in the outlined area of the plot. FIGS. 2A and 2B show FACS data for T cells and an irrelevant tumor antigen (+irr) and FIGS. 2C and 2D show FACS data for T cell and autologous tumor. FIGS. 2B and 2D show the FACS data for the break-down of the calcium aggregates circled in FIGS. 2A and 2C, respectively. As seen in FIG. 2D, tumor antigen-specific T cells were identified by using autologous tumor and Ca²⁺ dye.

FIGS. 3A-3F present FACS data for ovarian tumor cells that were stained with a violet cell tracker dye (surface stain) and an APC cell tracker dye (cell permeable calcium dye). The numbers in the quadrants represent the number of cells in the outlined area of the plot. FIG. 3A shows FACS data for dendritic cells alone, FIG. 3B shows FACS data for T cells with wild type peptide dendritic cells, FIG. 3C shows FACS data for T cells with mutant peptide dendritic cells, FIG. 3D shows FACS data for T cells alone, FIG. 3E shows FACS data for T cells with wild type peptide dendritic cells, and FIG. 3F shows FACS data for T cells with mutant peptide dendritic cells. FIGS. 3A, 3B, 3D, and 3E are plots of FACS data for cell tracker violet and forward scattered light (FSC). FIGS. 3C and 3F are plots of FACS data for calcium dyed cells over time (seconds). FIG. 3C is a plot of FACS data for the cells outlined in FIG. 3B and FIG. 3F is a plot of FACS data for the cells outlined in FIG. 3E.

FIGS. 4A-4F present FACS data for ovarian tumor cells that were stained with a violet cell tracker dye (surface stain) and an APC cell tracker dye (cell permeable calcium dye). The numbers in the quadrants represent the number of cells in the outlined area of the plot. FIG. 4A shows FACS data for dendritic cells alone (FCS), FIG. 3B shows a plot of FACS data for the cells outlined in FIG. 4A (calcium dyed cells over time), FIG. 4C shows FACS data for T cells alone (FCS), FIG. 4D shows a plot of FACS data for the cells outlined in FIG. 4C (calcium dyed cells over time), FIG. 4E shows FACS data for T cells with aCD3 (FCS), and FIG. 4F shows a plot of FACS data for the cells outlined in FIG. 4E (calcium dyed cells over time).

FIG. 5 presents FACS data for melanoma tumor cells that were stained with a violet cell tracker dye (surface stain) and an APC cell tracker dye (cell permeable calcium dye). As seen in the plot in the lower righthand corner, tumor antigen-specific T cells were identified by using autologous tumor and Ca²⁺ dye.

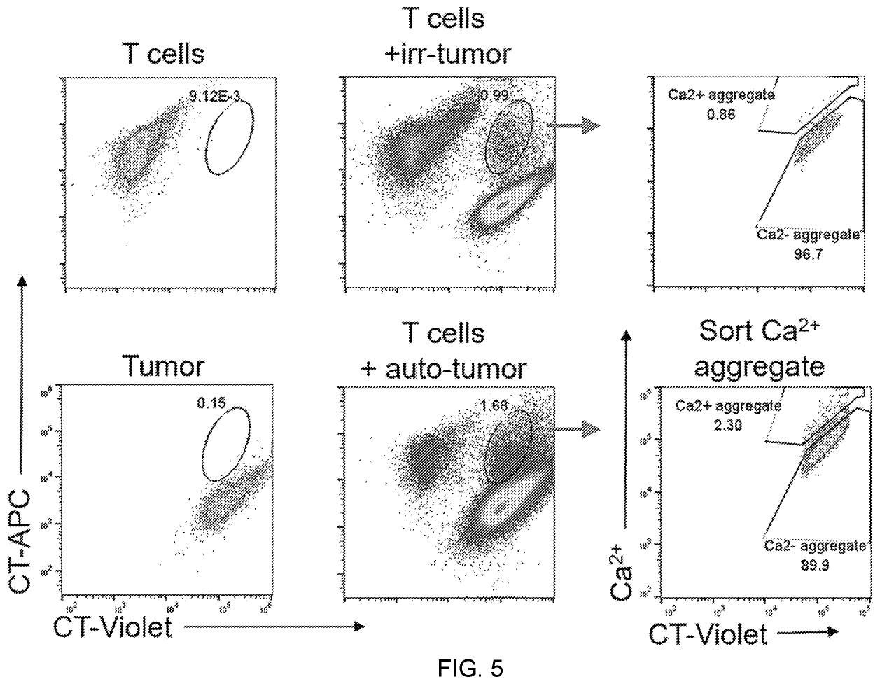

FIG. 6 presents FACS data for cells that were stained with a violet cell tracker dye and an APC cell tracker dye. Mock (no TCR) was the negative control and human NY-ESO was the positive control. “CM” is complete media only, “aCD3” is anti-CD3 (non-specifically) stimulated cells, “526” is a NY-ESO negative expressing tumor line, “624” is a NY-ESO positive expressing tumor line, “TC2650” is tumor cells exposed an irrelevant tumor (non-matched), and “TC3759” is tumor cells from patient 3759 from which melanoma cells were used to prepare TCR pairs. The cells were sorted by FACS to determine the cytokine release (TNFa and IFN-γ) after being cultured for one week with the tumor cells and then exposed to GOLGISTOP™ protein transport inhibitor and then stained. GOLGISTOP™ in this assay effectively prevented the cytokines produced by the cells to be trapped inside the cells so that accurate cytokine release rates can be visualized by FACS.

FIG. 7 presents FACS data for cells that were stained with a violet cell tracker dye and an APC cell tracker dye. The results for TCR pair 3759-A1 is shown on the top and TCR pair 3759-A3 is shown on the bottom. “PMA/ION” is phorbol myristate acetate/ionomycin and was used as a control because it stimulates the cells but bypasses the immune system stimulation.

FIG. 8 presents FACS data for cells that were stained with a violet cell tracker dye and an APC cell tracker dye. The results for TCR pair 3759-A4 is shown on the top and TCR pair 3759-A12 is shown on the bottom.

FIG. 9 is a graph showing the level of TCR pairs present in the blood of patient 3759 one month after receving an infusion containing the TCR pairs of FIGS. 7 and 8.

FIG. 10 presents FACS showing calcium flux over time in CD4⁺ (left) and CD8⁺ (right) cells. The cells were manipulated by knocking out Cish (top line) and bottom line was control.

### DETAILED DESCRIPTION OF THE INVENTION

It has been discovered that quickly identifying tumor-specific T cells, and isolating their T cell receptors (TCRs), may provide any one or more of a variety of advantages. These advantages may include, for example, decreased time until patient treatment, greater likelihood of treatment successful due to the use of tumor specific TCRs, and greatly decreased cost of treatment due to reduced processing resources (as compared to the current methods used to produce cancer-reactive T cells).

An embodiment of the invention provides a method of producing an isolated population of T cells. The method may comprise providing a tumor sample containing T cells and tumor cells from a patient having a tumor. The tumor sample can be any suitable tumor sample (liquid or solid) that has T cells present in a sufficient quantity to produce at least one TCR for sequencing. The tumor sample may be obtained by, for example, resection, blood draw, leukapheresis, or another suitable technique.

The method may further comprise separating the T cells from the tumor cells of the tumor sample to produce a separated population of T cells and a separated population of tumor cells. This separation step may be accomplished using any suitable technique that detects intracellular Ca²⁺ release. For example, FACS, magnetic separation (MACs), acoustic separation, and electrokinetic separation. This separation step relies on sorting based on the amount and detection of intracellular Ca²⁺ release via dye, recombinant protein, and/or Ca²⁺ reporter element (see e.g., Shield I V et al.. Lab Chip, 15: 1230 (2015)). Intracellular Ca²⁺ release occurs during aggregate formation with target tumor cells or APCs. Preferably the separating is carried out using FACS, as FACs provides reliable output.

The population of T cells may include any type of T cells. The T cell may be a human T cell. The T cell can be any type of T cell and can be of any developmental stage, including but not limited to, CD4⁺/CD8⁺ double positive T cells, CD4⁺ T cells, e.g., Th₁ and Th₂ cells, CD8⁺ T cells (e.g., cytotoxic T cells), Th₉ cells, TIL, memory T cells, naïve T cells, and the like. The T cell may be a CD8⁺ T cell or a CD4⁺ T cell. In a preferred embodiment, the T cells are tumor infiltrating lymphocytes (TIL).

The method may comprise exposing the population of T cells separated from the tumor cells to at least one non-cytotoxic cell permeable Ca²⁺ dye to dye the T cells. The cell permeable Ca²⁺ dye may be any suitable Ca²⁺ dye that fluoresces in the presence of Ca²⁺ and is capable of crossing the cell membrane of the T cell, for example, a Ca²⁺ dye comprising 4-(6-acetoxymethoxy-2,7-dichloro-3-oxo-9-xanthenyl)-4′-methyl-2,2′(ethylenedioxy)dianiline-N,N,N′,N′-tetraacetic acid tetrakis(acetoxymethyl) ester (e.g., Fluo3-AM, Thermo-Fisher Scientific), glycine, N-[2-[(acetyloxy)methoxy]-2-oxoethyl]-N-[5-[2-[2-[bis[2-[(acetyloxy)methoxy]-2-oxoethyl]amino]-5-methylphenoxy]ethoxy]-2-[(5-oxo-2-thioxo-4-imidazolidinylidene)methyl]-6-benzofuranyl] (e.g., FuraRed-AM, Thermo-Fisher Scientific), glycine, N-[2-[(acetyloxy)methoxy]-2-oxoethyl]-N-[2-[2-[5-[[[3′,6′-bis(acetyloxy)-2′,7′-dichloro-3-oxospiro[isobenzofuran-1(3H),9′-[9H]xanthen]-5-yl]carbonyl]amino]-2-[bis[2-[(acetyloxy)methoxy]-2-oxoethyl]amino]phenoxy]ethoxy]phenyl]-, (acetyloxy)methyl ester (e.g., CALCIUM GREEN, Thermo-Fisher Scientific), xanthylium, 9-[4-[[[[4-[bis[2-[(acetyloxy)methoxy]-2-oxoethyl]amino]-3-[2-[2-[bis[2-[(acetyloxy)methoxy]-2-oxoethyl]amino]phenoxy]ethoxy]phenyl]amino]thioxomethyl]amino]-2-carboxyphenyl]-3,6-bis(dimethylamno)-, inner salt (e.g., CALCIUM ORANGE, Thermo-Fisher Scientific),

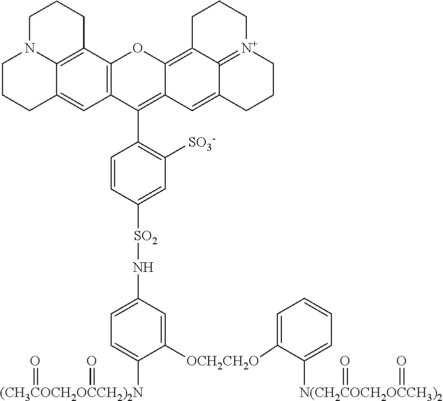

(e.g., CALCIUM CRIMSON, Thermo-Fisher Scientific), glycine, N-[4-[6-[(acetyloxy)methoxy]-2,7-dichloro-3-oxo-3H-xanthen-9-yl]-2-[2-[2-[bis[2-[(acetyloxy)methoxy]-2-oxyethyl]amino]-5-methylphenoxy]ethoxy]phenyl]-N-[2-[(acetyloxy)methoxy]-2-oxyethyl]-, (acetyloxy)methyl ester 121714-22-5 (e.g., FLUO-3, Thermo-Fisher Scientific), glycine, N-[4-[6-[(acetyloxy)methoxy]-2,7-difluoro-3-oxo-3H-xanthen-9-yl]-2-[2-[2-[bis[2-[(acetyloxy)methoxy]-2-oxoethyl]amino]-5-methylphenoxy]ethoxy]phenyl]-N-[2-[(acetyloxy)methoxy]-2-oxoethyl]-(acetyloxy)methyl ester (e.g., FLUO-4, Thermo-Fisher Scientific), 5-oxazolecarboxylic acid, 2-(6-(bis(2-((acetyloxy)methoxy)-2-oxoethyl)amino)-5-(2-(2-(bis(2-((acetyloxy)methoxy)-2-oxoethyl)amino)-5-methylphenoxy)ethoxy)-2-benzofuranyl)-, (acetyloxy)methyl ester (e.g., FURA-2, Thermo-Fisher Scientific), 1H-indole-6-carboxylic acid, 2-[4-[bis[2-[(acetyloxy)methoxy]-2-oxoethyl]amino]-3-[2-[2-[bis[2-[(acetyloxy)methoxy]-2-oxoetyl]amino]-5-methylphenoxy]ethoxy]phenyl]-, (acetyloxy)methyl ester (e.g., Indo-1, Thermo-Fisher Scientific), glycine,N-[2-[[8-[bis(carboxymethyl)amino]-6-methoxy-2-quinolinyl]methoxy]-4-methylphenyl]-N-(carboxymethyl)-potassium salt (e.g., Quin-2, Sigma-Aldrich), bis(acetoxymethyl) 2,2′-((4-(6-(acetoxymethoxy)-3-oxo-3H-xanthen-9-yl)-2-(2-(bis(2-acetoxymethoxy)-2-oxoethyl)amino)phenoxy)ethoxy)phenyl)azanediyl)diacetate (e.g., Fluo-8, Thermo-Fisher Scientific), FLUO-FORTE calcium dye (Enzo Life Sciences),

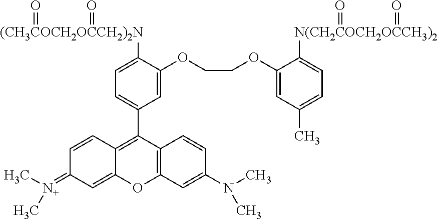

(e.g., Rhod-2, Thermo-Fisher Scientific), Rhod-3 (Thermo-Fisher Scientific), glycine, N-[2-[(acetyloxy)methoxy]-2-oxoethyl]-N-[4-[[[3′,6′-bis(acetyloxy)-2′,7′-difluoro-3-oxospiro[isobenzofuran-1(3H),9′-[9H]xanthen]-5-yl]carbonyl]amino]-2-[2-[2-[bis[2-[(acetyloxy)methoxy]-2-oxoethyl]amino]phenoxy]ethoxy]phenyl]-, (acetyloxy)methyl ester (e.g., OREGON GREEN BAPTA-1, Thermo-Fisher Scientific), and

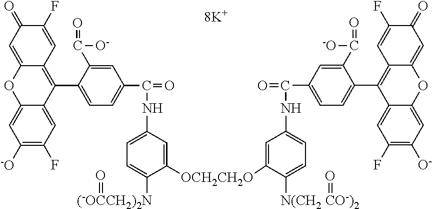

(e.g., OREGON GREEN BAPTA-2, Thermo-Fisher Scientific). In general, calcium indicators are unable to cross lipid membranes due to having a charged carboxy group. Therefore, physical or chemical methods are needed to load them into the cell. Acetoxymethyl (AM) esters protect the carboxylic groups as AM esters make the dyes neutral so they can cross the cell membrane. Once inside the cell, esterases cleave the AM groups. The Ca²⁺ dyed T cells will be distinguishable during sorting from other cell types and non-Ca²⁺ dyed T cells.

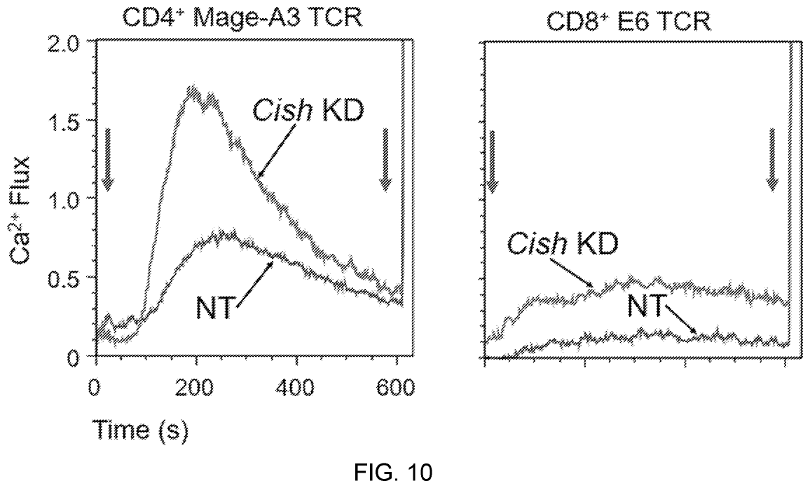

In an embodiment, the separated T cells can be manipulated by knocking out Cish, a member of the suppressor of cytokine signaling (SOCS) family. Cish is induced by TCR stimulation in CD8⁺ T cells and inhibits their functional avidity against tumors. Knockout of Cish (e.g., genetic deletion of Cish) in CD8⁺ T cells enhances their expansion, functional avidity, and cytokine polyfunctionality, resulting in pronounced and durable regression of established tumors (see Palmer et al., J. Exp. Med., 212(12): 2095-2113 (2015)). As seen in FIG. 10, knockout of Cish makes T cells containing tumor-reactive TCRs have increased peak calcium levels. Therefore, knocking out Cish in the separated T cells will increase calcium levels and increase the visibility of the at least one non-cytotoxic cell permeable Ca²⁺ dye within the dyed T cells.

The method may also comprise exposing target cells to at least one non-cytotoxic cell membrane dye to produce dyed target cells. The cell membrane dye may be any suitable cell membrane dye that fluoresces when the dye is bound to the cell membrane of a cell, does not interfere with the Ca²⁺ dye (i.e., does not spectrally overlap with the Ca²⁺ dye), and is non-cytotoxic, for example, an organic dye that excites with ultraviolet (355 nm) or violet (405 nm) laser (e.g., EFLOUR450™ violet dye, ThermoFisher Scientific) and/or carboxyfluorescein succinimidyl ester (CFSE, Sigma-Aldrich), CytoPainter Green, Red, Blue, or Orange (Abcam PLC), CELLTRACKER Blue, Orange, Red, or Deep Red 5-chloromethylfluorescein diacetate (CMFDA, Sigma-Aldrich), and QTRACKER labels (e.g. 525, 565, 585, 605, 625, 655, 705, and 800 nm, ThermoFisher Scientific).

In an embodiment of the invention, the method comprises the use of target cells. Target cells can be cells from a patient's or donor's tumor (solid or liquid, single cells or aggregates thereof) or antigen presenting cells (APCs). The target cells that are derived from a tumor express one or more tumor antigens. The APCs may be loaded or genetically modified to express one or more tumor antigens. Suitable APCs include peripheral blood cells, such as peripheral blood mononuclear cells (PBMCs), such as peripheral blood lymphocytes (PBLs), B cells, and dendritic cells. Alternatively, target cells can be generated using in vitro generated tumor lines, T-cell depleted dissociated tumor resections, and magnetic-bead fractionation.

In another embodiment of the invention, the method comprises exposing the dyed T cells to the dyed target cells under conditions sufficient for at least a portion of the dyed T cells to specifically bind to the one or more tumor antigens of the dyed target cells. This exposure step can be completed using any suitable technique and conditions in which a sufficient amount of binding may occur.

In another embodiment of the invention, the method comprises identifying the dyed T cells which exhibit both (i) specific binding to the dyed target cells and (ii) absorption of a level of the at least one cell permeable Ca²⁺ dye sufficient to indicate T cell receptor activation. For this step, FACS may be used, or another suitable technique. The absorption level that is sufficient to indicate T cell receptor activation may be determined, e.g., by running (1) a “control” of T cells alone and (2) T cells with “control target cells” prior to setting up the capture gates. The gate may be set so less than about 1% of aggregate+calcium dyed cells are captured. The gating may be determined based on the frequency of T cell-tumor interaction. For example, the benchmark may be twice the background level. Depending on availability, the following can be run to determine the gating: (1) T cells alone, (2) T cells with empty APC, and/or (3) T cells plus irrelevant tumor (tumor without autologous tumor antigen). While there may be aggregation in (2) or (3), there will be minimal Ca²⁺ flux. The levels of (1), (2), and/or (3) can be used to set the gate to less than 1% of all events. The desired cells will be captured in the Ca²⁺ gate after positive T cell: relevant tumor/APC coculture. Appropriate control target cells include mismatched tumor cells or antigen presenting cells (APCs) either without peptide or with irrelevant peptide (non-targeted or non-mutated depending availability). Suitable APC's include autologous B cells, dendritic cells, and/or PBMCs. The APCs may be pulsed with the cancer antigen or a nucleotide sequence encoding the cancer antigen may be introduced into the APC.

In a further embodiment of the invention, the method comprises separating the dyed T cells identified to exhibit (i) specific binding to the dyed target cells and (ii) absorption of a level of the at least one cell permeable Ca²⁺ dye sufficient to indicate T cell receptor activation from the cells that do not exhibit both (i) and (ii). For this step, FACS may be used, or another suitable technique. The separated cells can be sorted into a container, for example, a PCR plate.

In an embodiment of the invention, the method comprises obtaining a sequence of a TCR from a T cell which exhibits (i) specific binding to the dyed target cells and (ii) absorption of a level of the at least one cell permeable Ca²⁺ dye sufficient to indicate T cell receptor activation. For this step, nested PCR or alignment by adaptive screening may be used, or another suitable technique.

In an embodiment of the invention, the method comprises inserting the sequence of the T cell receptor into PBMC to provide an isolated population of cells for adoptive cell therapy. For this step, the following techniques may be used: (1) using a retroviral vector as described in, for example, Johnson et al., Blood, 114: 535-546 (2009); (2) using targeted integration as described in, for example, Roth et al., Nature, 559: 405-409 (2018); (3) using a transposon as described in, for example, Peng et al., Gene Ther., 16: 1042-1049 (2009); and using transiently expressed RNA (e.g., mRNA) as described in, for example, Zhao et al., Mol. Ther., 13: 151-159 (2006), or another suitable technique. In an embodiment, PBMC are transduced with a vector comprising the sequence of the T cell receptor to provide the isolated population of T cells for adoptive cell therapy. While the PBMC may be allogeneic, in a preferred embodiment, the PBMC are autologous to the patient.

The PBMC used for to provide an isolated population of cells for adoptive cell therapy can be any suitable PBMC, for example, a lymphocyte (e.g., a T cell or a B cell) or a monocyte. In a preferred embodiment, the PBMC is a T cell.

In an embodiment of the invention, the method allows for a patient to receive a population of cells for ACT (with TCRs specific for the patient's tumor) only about 30 or fewer days after a tumor sample is removed from the patient. For example, the patient may receive a population of cells for ACT (with TCRs specific for the patient's tumor) only about 28 or fewer, about 26 or fewer, about 24 or fewer, about 22 or fewer, about 20 or fewer, about 18 or fewer, about 16 or fewer, about 15 or fewer, about 14 or fewer, about 13 or fewer, about 12 or fewer, about 11 or fewer, about 10 or fewer, about 9 or fewer, about 8 or fewer, about 7 or fewer, about 6 or fewer, about 5 or fewer, about 4 or fewer, about 3 or fewer, or about 2 or fewer days after a tumor sample is removed from the patient.

In an embodiment of the invention, the method provides a ratio of dyed T cells to dyed target cells. This ratio can be from about 1:1 to about 1:100 of dyed T cells to dyed target cells. For example, the ratio can be from about 1:1 to about 1:75, about 1:1 to about 1:50, about 1:1 to about 1:25, about 1:1 to about 1:20, about 1:1 to about 1:15, about 1:1 to about 1:10, about 1:1 to about 1:5, or about 1:5 to about 1:10. In this regard, the number of dyed T cells to dyed target cells can be from about 1×10⁶/mL to about 5×10⁶/ml. In this regard, the amount of dyed T cells can be from about 1×10⁶/mL to about 100×10⁶/ml, from about 1×10⁶/mL to about 75×10⁶/ml, from about 1×10⁶/mL to about 50×10⁶/ml, from about 1×10⁶/mL to about 25×10⁶/ml, from about 1×10⁶/mL to about 20×10⁶/ml, from about 1×10⁶/mL to about 15×10⁶/ml, from about 1×10⁶/mL to about 10×10⁶/ml, or from about 1×10⁶/mL to about 5×10⁶/ml, respectively. In this regard, the amount of dyed target cells can be from about 1×10⁶/mL to about 100×10⁶/ml, from about 1×10⁶/mL to about 75×10⁶/ml, from about 1×10⁶/mL to about 50×10⁶/ml, from about 1×10⁶/mL to about 25×10⁶/ml, from about 1×10⁶/mL to about 20×10⁶/ml, from about 1×10⁶/mL to about 15×10⁶/ml, from about 1×10⁶/mL to about 10×10⁶/ml, or from about 1×10⁶/mL to about 5×10⁶/ml, respectively.

In an embodiment of the invention, the TCRs of the T cells have antigenic specificity for a tumor (i.e., cancer antigen) of the dyed target cells. In a further embodiment of the invention, the TCRs of the T cells specifically bind to the one or more tumor antigens of the dyed target cells. The terms “cancer antigen” and “tumor antigen,” as used herein, refers to any molecule (e.g., protein, polypeptide, peptide, lipid, carbohydrate, etc.) solely or predominantly expressed or over-expressed by a tumor cell or cancer cell, such that the antigen is associated with the tumor or cancer. The cancer antigen can additionally be expressed by normal, non-tumor, or non-cancerous cells. However, in such cases, the expression of the cancer antigen by normal, non-tumor, or non-cancerous cells is not as robust as the expression by tumor or cancer cells. In this regard, the tumor or cancer cells can over-express the antigen or express the antigen at a significantly higher level, as compared to the expression of the antigen by normal, non-tumor, or non-cancerous cells. Also, the cancer antigen can additionally be expressed by cells of a different state of development or maturation. For instance, the cancer antigen can be additionally expressed by cells of the embryonic or fetal stage, which cells are not normally found in an adult host. Alternatively, the cancer antigen can be additionally expressed by stem cells or precursor cells, which cells are not normally found in an adult host.

The cancer antigen can be an antigen expressed by any cell of any cancer or tumor, including the cancers and tumors described herein. The cancer antigen may be a cancer antigen of only one type of cancer or tumor, such that the cancer antigen is associated with or characteristic of only one type of cancer or tumor. Alternatively, the cancer antigen may be a cancer antigen (e.g., may be characteristic) of more than one type of cancer or tumor. For example, the cancer antigen may be expressed by both breast and prostate cancer cells and not expressed at all by normal, non-tumor, or non-cancer cells. Cancer antigens are known in the art and include, for instance, CXorf61, mesothelin, CD19, CD22, CD276 (B7H3), gp100, MART-1, Epidermal Growth Factor Receptor Variant III (EGFRVIII), TRP-1, TRP-2, tyrosinase, NY-ESO-1 (also known as CAG-3), MAGE-1, MAGE-3, etc.

The cancer may be any cancer, including any of acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, cholangiocarcinoma, colon cancer, esophageal cancer, cervical cancer, gastrointestinal carcinoid tumor, Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer (e.g., renal cell carcinoma (RCC)), small intestine cancer, soft tissue cancer, stomach cancer, testicular cancer, thyroid cancer, ureter cancer, and urinary bladder cancer. In certain preferred embodiments, the antigen-specific receptor has specificity for a melanoma antigen. In certain preferred embodiments, the antigen-specific receptor has specificity for an ovarian cancer antigen.

In an embodiment of the invention, the cancer antigen is a cancer neoantigen. A cancer neoantigen is an immunogenic mutated amino acid sequence which is encoded by a cancer-specific mutation. Cancer neoantigens are not expressed by normal, non-cancerous cells and may be unique to the patient. ACT with T cells which have antigenic specificity for a cancer neoantigen may provide a “personalized” therapy for the patient.

In an embodiment of the invention, the antigen-specific receptor is a T-cell receptor (TCR). A TCR generally comprises two polypeptides (i.e., polypeptide chains), such as α-chain of a TCR, a β-chain of a TCR, a γ-chain of a TCR, a δ-chain of a TCR, or a combination thereof. Such polypeptide chains of TCRs are known in the art. The antigen-specific TCR can comprise any amino acid sequence, provided that the TCR can specifically bind to and immunologically recognize an antigen, such as a cancer antigen or epitope thereof.

The T cell can comprise and express an endogenous TCR, i.e., a TCR that is endogenous or native to (naturally-occurring on) the T cell. In such a case, the T cell comprising the endogenous TCR can be a T cell that was isolated from a patient which is known to express the particular cancer antigen. In certain embodiments, the T cell is a primary T cell isolated from a patient afflicted with cancer. In some embodiments, the cell is a TIL or a T cell isolated from a human cancer patient.

In some embodiments, the patient from which a cell is isolated is immunized with an antigen of, or specific for, a cancer. The patient may be immunized prior to obtaining the cell from the patient. In this way, the isolated cells can include T cells induced to have specificity for the cancer to be treated, or can include a higher proportion of cells specific for the cancer.

Alternatively, a T cell comprising and expressing an endogenous antigen-specific TCR can be a T cell within a mixed population of cells isolated from a patient, and the mixed population can be exposed to the antigen which is recognized by the endogenous TCR while being cultured in vitro. In this manner, the T cell which comprises the TCR that recognizes the cancer antigen expands or proliferates in vitro, thereby increasing the number of T cells having the endogenous antigen-specific TCR.

The TCR sequence can be inserted into PBMC to provide an isolated population of cells for adoptive cell therapy. In this regard, the nucleic acids may be introduced into the cell using any suitable method such as, for example, transfection, transduction, or electroporation. For example, cells can be transduced with viral vectors using viruses (e.g., retrovirus or lentivirus) and cells can be transduced with transposon vectors using electroporation. In an embodiment, the PBMC are transduced with a vector comprising the sequence of the T cell receptor to provide the isolated population of cells for adoptive cell therapy.

The terms “nucleic acid” and “polynucleotide,” as used herein, refer to a polymeric form of nucleotides of any length, either ribonucleotides (RNA) or deoxyribonucleotides (DNA). These terms refer to the primary structure of the molecule, and thus include double- and single-stranded DNA, double- and single-stranded RNA, and double-stranded DNA-RNA hybrids. The terms include, as equivalents, analogs of either RNA or DNA made from nucleotide analogs and modified polynucleotides such as, though not limited to, methylated and/or capped polynucleotides. In an embodiment of the invention, the nucleic acid is complementary DNA (cDNA).

The term “nucleotide” as used herein refers to a monomeric subunit of a polynucleotide that consists of a heterocyclic base, a sugar, and one or more phosphate groups. The naturally occurring bases (guanine (G), adenine (A), cytosine (C), thymine (T), and uracil (U)) are typically derivatives of purine or pyrimidine, though the invention includes the use of naturally and non-naturally occurring base analogs. The naturally occurring sugar is the pentose (five-carbon sugar) deoxyribose (which forms DNA) or ribose (which forms RNA), though the invention includes the use of naturally and non-naturally occurring sugar analogs. Nucleic acids are typically linked via phosphate bonds to form nucleic acids or polynucleotides, though many other linkages are known in the art (e.g., phosphorothioates, boranophosphates, and the like). Methods of preparing polynucleotides are within the ordinary skill in the art (Green and Sambrook, Molecular Cloning: A Laboratory Manual, (4th Ed.) Cold Spring Harbor Laboratory Press, New York (2012)).

The nucleic acids described herein can be incorporated into a recombinant expression vector. For purposes herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors may not be naturally-occurring as a whole. However, parts of the vectors can be naturally-occurring. The recombinant expression vectors can comprise any type of nucleotides, including, but not limited to DNA and RNA, which can be single-stranded or double-stranded, synthesized or obtained in part from natural sources, and which can contain natural, non-natural or altered nucleotides. The recombinant expression vectors can comprise naturally-occurring or non-naturally-occurring intemucleotide linkages, or both types of linkages. Preferably, the non-naturally occurring or altered nucleotides or intemucleotide linkages do not hinder the transcription or replication of the vector. Examples of recombinant expression vectors that may be useful in the inventive methods include, but are not limited to, plasmids, viral vectors (retroviral vectors, gamma-retroviral vectors, or lentiviral vectors), and transposons. The vector may then, in turn, be introduced into the cells by any suitable technique such as, e.g., gene editing, transfection, transformation, or transduction as described, for example, Green and Sambrook, Molecular Cloning: A Laboratory Manual (4^{th} Ed.), Cold Spring Harbor Laboratory Press (2012). Many transfection techniques are known in the art and include, for example, calcium phosphate DNA co-precipitation; DEAE-dextran; electroporation; cationic liposome-mediated transfection; tungsten particle-facilitated microparticle bombardment; and strontium phosphate DNA co-precipitation. Phage or viral vectors can be introduced into host cells, after growth of infectious particles in suitable packaging cells, many of which are commercially available.

In an embodiment of the invention, the method further comprises expanding the number of cells in the presence of one or both of (a) one or more cytokines and (b) one or more non-specific T cell stimuli. Examples of non-specific T cell stimuli include, but are not limited to, one or more of irradiated allogeneic feeder cells, irradiated autologous feeder cells, anti-CD3 antibodies (e.g., OKT3 antibody), anti-4-1BB antibodies, and anti-CD28 antibodies. In preferred embodiment, the non-specific T cell stimulus may be anti-CD3 antibodies and anti-CD28 antibodies conjugated to beads. Any one or more cytokines may be used in the inventive methods. Exemplary cytokines that may be useful for expanding the numbers of cells include interleukin (IL)-2, IL-7, IL-21, IL-15, or a combination thereof.

Expansion of the numbers of cells can be accomplished by any of a number of methods as are known in the art as described in, for example, U.S. Pat. Nos. 8,034,334; 8,383,099; and U.S. Patent Application Publication No. 2012/0244133. For example, expansion of the numbers of cells may be carried out by culturing the cells with OKT3 antibody, IL-2, and feeder PBMC (e.g., irradiated allogeneic PBMC).

An embodiment of the invention further provides an isolated or purified population of T cells produced by any of the inventive methods described herein. The populations of T cells produced by the inventive methods may provide any one or more of many advantages.

The population of cells produced by according to the inventive methods can be a heterogeneous population comprising the cells described herein, in addition to at least one other cell, e.g., a cell other than a T cell, e.g., a B cell, a macrophage, a neutrophil, an erythrocyte, a hepatocyte, an endothelial cell, an epithelial cell, a muscle cell, a brain cell, etc. Alternatively, the population of cells produced by the inventive methods can be a substantially homogeneous population, in which the population comprises mainly of the cells, e.g., T cells described herein. The population also can be a clonal population of cells, in which all cells of the population are clones of a single cell, e.g., T cell. In one embodiment of the invention, the population of cells is a clonal population comprising cells, e.g., T cells comprising a recombinant expression vector encoding the antigen-specific receptor as described herein.

The inventive isolated or purified population of cells produced according to the inventive methods may be included in a composition, such as a pharmaceutical composition. In this regard, an embodiment of the invention provides a pharmaceutical composition comprising the isolated or purified population of cells described herein and a pharmaceutically acceptable carrier.

Preferably, the carrier is a pharmaceutically acceptable carrier. With respect to pharmaceutical compositions, the carrier can be any of those conventionally used for the administration of cells. Such pharmaceutically acceptable carriers are well-known to those skilled in the art and are readily available to the public. It is preferred that the pharmaceutically acceptable carrier be one which has no detrimental side effects or toxicity under the conditions of use.

The choice of carrier will be determined in part by the particular method used to administer the population of cells. Accordingly, there are a variety of suitable formulations of the pharmaceutical composition of the invention. Suitable formulations may include any of those for parenteral, subcutaneous, intravenous, intramuscular, intraarterial, intrathecal, intratumoral, or interperitoneal administration. More than one route can be used to administer the population of cells, and in certain instances, a particular route can provide a more immediate and more effective response than another route.

Preferably, the population of cells is administered by injection, e.g., intravenously. A suitable pharmaceutically acceptable carrier for the cells for injection may include any isotonic carrier such as, for example, normal saline (about 0.90% w/v of NaCl in water, about 300 mOsm/L NaCl in water, or about 9.0 g NaCl per liter of water), NORMOSOL electrolyte solution (Abbott, Chicago, Ill.), PLASMA-LYTE A (Baxter, Deerfield, Ill.), about 5% dextrose in water, or Ringer's lactate. In an embodiment, the pharmaceutically acceptable carrier is supplemented with human serum albumen.

The T cells administered to the patient can be allogeneic or autologous to the patient. In “autologous” administration methods, cells are removed from a patient, stored (and optionally modified), and returned back to the same patient. In “allogeneic” administration methods, a patient receives cells from a genetically similar, but not identical, donor. Preferably, the T cells are autologous to the patient. Autologous cells may, advantageously, reduce or avoid the undesirable immune response that may target an allogeneic cell such as, for example, graft-versus-host disease.

In the instance that the T cell(s) are autologous to the patient, the patient can be immunologically naïve, immunized, diseased, or in another condition prior to isolation of the cell(s) from the patient. In some instances, it is preferable for the method to comprise immunizing the patient with an antigen of the cancer prior to isolating the T cell(s) from the patient, introducing nucleic acid into the cell(s), and the administering of the T cell(s) or composition thereof.

In accordance with an embodiment of the invention, a patient with cancer can be therapeutically immunized with an antigen from, or associated with, that cancer, including immunization via a vaccine. While not desiring to be bound by any particular theory or mechanism, the vaccine or immunogen is provided to enhance the patient's immune response to the cancer antigen present in the cancerous tissue. Such a therapeutic immunization includes, but is not limited to, the use of recombinant or natural cancer proteins, peptides, or analogs thereof, or modified cancer peptides, or analogs thereof that can be used as a vaccine therapeutically as part of adoptive immunotherapy. The vaccine or immunogen, can be a cell, cell lysate (e.g., from cells transfected with a recombinant expression vector), a recombinant expression vector, or antigenic protein or polypeptide. Alternatively, the vaccine, or immunogen, can be a partially or substantially purified recombinant cancer protein, polypeptide, peptide or analog thereof, or modified proteins, polypeptides, peptides or analogs thereof. The protein, polypeptide, or peptide may be conjugated with lipoprotein or administered in liposomal form or with adjuvant. Preferably, the vaccine comprises one or more of (i) the cancer antigen for which the antigen-specific receptor has antigenic specificity, (ii) an epitope of the antigen, and (iii) a vector encoding the antigen or the epitope.

For purposes of the invention, the dose, e.g., number of cells administered should be sufficient to effect, e.g., a therapeutic or prophylactic response, in the patient over a reasonable time frame. For example, the number of cells administered should be sufficient to bind to a cancer antigen or treat or prevent cancer in a period of from about 2 hours or longer, e.g., 12 to 24 or more hours, from the time of administration. In certain embodiments, the time period could be even longer. The number of cells administered will be determined by, e.g., the efficacy of the particular population of cells to be administered and the condition of the animal (e.g., human), as well as the body weight of the animal (e.g., human) to be treated.

Many assays for determining an administered number of cells are known in the art. For purposes of the invention, an assay, which comprises comparing the extent to which target cells are lysed or one or more cytokines such as, e.g., IFN-γ and IL-2 is secreted upon administration of a given number of such cells to a patient among a set of patients of which is each given a different number of the cells, e.g., T cells, could be used to determine a starting number to be administered to a patient. The extent to which target cells are lysed or cytokines such as, e.g., IFN-γ and IL-2 are secreted upon administration of a certain number can be assayed by methods known in the art. Secretion of cytokines such as, e.g., IL-2, may also provide an indication of the quality (e.g., phenotype and/or effectiveness) of a T cell preparation.

The number of cells administered also will be determined by the existence, nature and extent of any adverse side effects that might accompany the administration of a particular population of cells. Typically, the attending physician will decide the number of cells with which to treat each individual patient, taking into consideration a variety of factors, such as age, body weight, general health, diet, sex, route of administration, and the severity of the condition being treated. By way of example and not intending to limit the invention, the number of cells, e.g., T cells, to be administered can be about 10×10⁶ to about 10×10¹¹ cells per infusion, about 10×10⁹ cells to about 10×10¹¹ cells per infusion, or 10×10⁷ to about 10×10⁹ cells per infusion.

It is contemplated that the populations of T cells produced according to the inventive methods can be used in methods of treating or preventing cancer in a patient. In this regard, an embodiment of the invention provides a method of treating or preventing cancer in a patient, comprising (i) administering cells to the patient according to any of the methods described herein; (ii) administering to the patient the cells produced according to any of the methods described herein; or (iii) administering to the patient any of the isolated populations of cells or pharmaceutical compositions described herein; in an amount effective to treat or prevent cancer in the patient.

In an embodiment of the invention, the method of treating or preventing cancer may comprise administering the cells or pharmaceutical composition to the patient in an amount effective to reduce metastases in the patient. For example, the inventive methods may reduce metastatic nodules in the patient.

One or more additional therapeutic agents can be co-administered to the patient. Use of “co-administering” herein means administering one or more additional therapeutic agents and the isolated population of cells sufficiently close in time such that the isolated population of cells can enhance the effect of one or more additional therapeutic agents, or vice versa. In this regard, the isolated population of cells can be administered first and the one or more additional therapeutic agents can be administered second, or vice versa. Alternatively, the isolated population of cells and the one or more additional therapeutic agents can be administered simultaneously. Additional therapeutic agents that may enhance the function of the isolated population of cells may include, for example, one or more cytokines or one or more antibodies (e.g., antibodies that inhibit PD-1 function). An exemplary therapeutic agent that can be co-administered with the isolated population of cells is IL-2. Without being bound to a particular theory or mechanism, it is believed that IL-2 may enhance the therapeutic effect of the isolated population of cells, e.g., T cells.

An embodiment of the invention further comprises lymphodepleting the patient prior to administering the isolated population of cells. Examples of lymphodepletion include, but may not be limited to, nonmyeloablative lymphodepleting chemotherapy, myeloablative lymphodepleting chemotherapy, total body irradiation, etc.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of treatment or prevention of cancer in a mammal. Furthermore, the treatment or prevention provided by the inventive method can include treatment or prevention of one or more conditions or symptoms of the disease, e.g., cancer, being treated or prevented. Also, for purposes herein, “prevention” can encompass delaying the onset or recurrence of the disease, or a symptom or condition thereof.

The term “isolated,” as used herein, means having been removed from its natural environment. The term “purified,” as used herein, means having been increased in purity, wherein “purity” is a relative term, and not to be necessarily construed as absolute purity. For example, the purity can be at least about 50%, can be greater than about 60%, about 70% or about 80%, about 90% or can be about 100%.

Unless stated otherwise, as used herein, the term “patient” refers to any mammal including, but not limited to, mammals of the order Logomorpha, such as rabbits; the order Carnivora, including Felines (cats) and Canines (dogs); the order Artiodactyla, including Bovines (cows) and Swines (pigs); or of the order Perssodactyla, including Equines (horses). It is preferred that the mammals are non-human primates, e.g., of the order Primates, Ceboids, or Simoids (monkeys) or of the order Anthropoids (humans and apes). In some embodiments, the mammal may be a mammal of the order Rodentia, such as mice and hamsters. In other embodiments, the mammal is not a mouse. Preferably, the mammal is a non-human primate or a human. An especially preferred mammal is the human.

With respect to the inventive methods, the cancer can be any cancer, including any of the cancers described herein with respect to other aspects of the invention.

Examples of Non-Limiting Aspects of the Disclosure

Aspects, including embodiments, of the present subject matter described herein may be beneficial alone or in combination, with one or more other aspects or embodiments. Without limiting the foregoing description, certain non-limiting aspects of the disclosure numbered 1-20 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

1. A method of producing an isolated population of cells for adoptive cell therapy, the method comprising:

a) providing a tumor sample containing T cells and tumor cells from a patient having a tumor;

b) separating the T cells from the tumor cells of the tumor sample of a) to produce a separated population of T cells and a separated population of tumor cells;

c) exposing the separated population of T cells of b) to at least one non-cytotoxic cell permeable Ca²⁺ dye to produce dyed T cells;

d) exposing target cells to at least one non-cytotoxic cell membrane dye to produce dyed target cells, wherein the target cells are the separated population of tumor cells of b) or antigen presenting cells (APCs), wherein the separated population of tumor cells of b) express one or more tumor antigens and the APCs are loaded with or express one or more tumor antigens;

e) exposing the dyed T cells to the dyed target cells under conditions sufficient for at least a portion of the dyed T cells to specifically bind to the one or more tumor antigens of the dyed target cells;

f) identifying the dyed T cells which exhibit both (i) specific binding to the dyed target cells and (ii) absorption of a level of the at least one cell permeable Ca²⁺ dye sufficient to indicate T cell receptor activation;

g) separating the dyed T cells identified to exhibit both (i) and (ii) from dyed T cells which fail to exhibit both (i) and (ii);

h) obtaining a sequence of a T cell receptor from a T cell which exhibits both (i) and (ii); and

i) inserting the sequence of the T cell receptor of h) into peripheral blood mononuclear cells (PBMC) to provide an isolated population of cells for adoptive cell therapy.

2. The method according to aspect 1, wherein fluorescence-activated cell sorting (FACS) is used in f) and/or g).

3. The method according to aspect 1 or 2, wherein i) is completed in less than 7 days aftera).

4. The method according to any one of aspects 1-3, wherein the ratio of dyed T cells to dyed target cells in f) is from about 1:5 to about 1:10.

5. The method according to any one of aspects 1-4, wherein the T cell receptor of h) specifically binds to the one or more tumor antigens of the dyed target cells.

6. The method according to any one of aspects 1-5, wherein the PBMC are transduced with a vector comprising the sequence of the T cell receptor of h) to provide the isolated population of T cells for adoptive cell therapy.

7. The method according to aspect 6, wherein the vector is a retroviral vector.

8. The method according to any one of aspects 1-7, wherein the PBMC are autologous to the patient.

9. The method according to any one of aspects 1-8, further comprising culturing the PBMC in the presence of interleukin-2 (IL-2), interleukin-7 (IL-7), interleukin-15 (IL-15), interleukin-12 (IL-12), or a combination of two or more of the foregoing.

10. The method according to any one of aspects 1-9, wherein the patient has melanoma.

11. The method according to any one of aspects 1-10, wherein the patient has ovarian cancer.

12. The method according to any one of aspects 1-11, wherein the T cells of a) are tumor infiltrating lymphocytes (TIL).

13. The method of any one of aspects 1-12, wherein the cell membrane dye fluoresces when the dye is bound to the cell membrane of a cell.

14. The method of any one of aspects 1-13, wherein the at least one cell permeable Ca²⁺ dye fluoresces in the presence of Ca²⁺.

15. The method of any one of aspects 1-14, wherein the at least one cell permeable Ca²⁺ dye comprises 4-(6-acetoxymethoxy-2,7-dichloro-3-oxo-9-xanthenyl)-4′-methyl-2,2′(ethylenedioxy)dianiline-N,N,N′,N′-tetraacetic acid tetrakis(acetoxymethyl) ester.

16. The method of any one of aspects 1-14, wherein the at least one cell permeable Ca²⁺ dye comprises glycine, N-[2-[(acetyloxy)methoxy]-2-oxoethyl]-N-[5-[2-[2-[bis[2-[(acetyloxy)methoxy]-2-oxoethyl]amino]-5-methylphenoxy]ethoxy]-2-[(5-oxo-2-thioxo-4-imidazolidinylidene)methyl]-6-benzofuranyl].

17. An isolated population of T cells produced by the method according to any one of aspects 1-16.

18. A pharmaceutical composition comprising the isolated population of cells of aspect 17 and a pharmaceutically acceptable carrier.

19. A method of treating or preventing cancer in a patient, the method comprising producing an isolated T cell population according to the method of any one of aspects 1-16, and administering the isolated cell population, or a pharmaceutical composition comprising the isolated cell population, to the patient in an amount effective to treat or prevent cancer in the patient.

20. The T cell population isolated according to the method of any one of aspects 1-16, the population of aspect 17, or the composition of aspect 18, for use in the treatment or prevention of cancer in a patient.

The following examples further illustrate the invention but, of course, should not be construed as in any way limiting its scope.

### EXAMPLES

The following materials and methods were used in Examples 1-3.

The present methods involve T cells mixed with target cells in order to elicit a tumor-specific T cell response, and the isolation, identification and expression of tumor-specific T cell receptors in T cells. This is accomplished by staining T cells with Ca²⁺ sensitive dyes, surface staining target cells, and capturing tumor-specific aggregates using Ca²⁺ flux-based flow cytometry assay. The captured single T cell/target cell aggregates are TCR sequenced using nested PCR, and the TCRs are then expressed in autologous T cells using recombinant retroviruses, targeted integration, transposons, and/or transiently expressed RNA. Suitable methods for each step are described in further detail below.

T cells were enriched from tumor fragments, tumor cells, or peripheral blood lymphocytes using a suitable method. For example, T cells were grown out from tumor fragments, tumor cells, or dissociated tumors using favorable conditions such as T cell-centric cytokines (such as, for example IL-2, IL-7, IL-12, and/or IL-15) for several weeks. Alternatively, Pan-T cell magnetic enrichment protocols/kits were used on a mechanically disassociated tumor resection. A commercially available antibody ferrous-cocktail that binds to non-T cells was added, the magnetic field was applied, and untouched flow through cells were collected. The column-bound fraction was retained for target cell preparation (see below).

Target cells were then generated. Peripheral blood lymphocyte-derived APCs and neo-antigen were loaded using a suitable technique. For example, dendritic cells or B cell derived PBMCs were loaded using a suitable techniques. Alternatively, peptide pulse or tandem mini-gene electroporate APCs were loaded using suitable methods (e.g., Robbins et al., Nat. Med., 19: 747-752 (2013)). Target cells were also generated using tumor cells, for example, using in vitro generated tumor lines, T-depleted dissociated tumor resections, and from the previously described magnetic-bead fractionation.

Next, Ca²⁺ sensing-dyed T cells were combined with membrane stained target cells and immediately sorted into PCR-ready plates (e.g., a 96 well plate) as single aggregates using suitable techniques. First, T cells were prepared. The T cells were stained with a cell-permeable calcium dye (e.g., Flou3-AM and FuraRed-AM). At a cell concentration of 1×10⁶/mL in 2% fetal calf serum (FCS) and Hank's Balanced Salt Solution (HBSS) 4 μg/mL Flou3-AM and 10 μg/mL FuraRed-AM were added for 20 minutes in a light-protected incubator at 37° C. HBSS must have Ca²⁺ and Mg²⁺. The cells were washed again with 2% FCS HBSS buffer and then reconstituted at 2×10⁶/ml in 2% FCS and HBSS in 200 μl total volume.

Next, the target cells were prepared. The target cells were surface stained with a compatible cell tracker dye (i.e., a dye that minimizes spectral overlap with Flou3-AM (FITC) and FuraRed-AM (PE-APC)). Cell Tracker EFLOUR450™ violet dye was used at 1 μM for 10 minutes at 37° C. The cells were washed and reconstituted in 5×10⁶/ml in 2% FCS and HBSS in 200 ul.

The PCR plates were prepared by adding nested PCR mix (see e.g., Pasetto et al., Cancer Immunol. Res., 4: 734-743 (2016)). The single cell sorter was prepared and the cells were maintained at 37° C.

A T cell baseline was then established by using flow cytometry for 10 seconds. Single cell gates and Ca²⁺ dye gates were set. Flou3-AM was in the negative gate and FuraRed-AM was in the positive gate.

A target cell baseline was then established by using flow cytometry for 10 seconds. Single cell gates using EFLOUR450™ dyed positive cells were set. The aggregate gate was set in between both single gates and was double positive for FuraRed-AM and EFLOUR450™ dye. The sort gate was set to be aggregate positive and Flou3AM positive gate.

All of the cells and the collection chamber were then warmed to 37° C. T cells (200 μl) were added to the target cells (200 μl) and flow cytometry was immediately performed. The cells were sorted until the PCR plates were filled, or for 5 minutes. The PCR plates were replaced when full. The PCR plates were then covered and centrifuged. A ratio of 1 T cell to 5-10 target cells (1:5 to 1:10) yielded the maximal signal and the most reproducible results. Total cell yield varied depending on the quantity and composition of the source material and the volume was adjusted accordingly. A final density of 1×10⁶/mL T cells to 5-10×10⁶/ml target cells yielded optimal results.

Next, the TCRs were sequenced and were identified using a suitable technique, for example, by nested PCR or alignment by adaptive screening (see e.g., Pasetto et al., Cancer Immunol. Res., 4: 734-743 (2016)).

The TCRs were then cloned and produced (i.e., put into T cells) using a suitable technique. For example, the following techniques are suitable: (1) using a retroviral vector as described in, for example, Johnson et al., Blood, 114: 535-546 (2009); (2) using targeted integration as described in, for example, Roth et al., Nature, 559: 405-409 (2018); (3) using a transposon as described in, for example, Peng et al., Gene Ther., 16: 1042-1049 (2009); and using transiently expressed RNA (e.g., mRNA) as described in, for example, Zhao et al., Mol. Ther., 13: 151-159 (2006).

The cytokine release FACS data was prepared from cells that were cultured for one week and then exposed to GOLGISTOP™ protein transport inhibitor and then stained. GOLGISTOP™ in this assay effectively prevented the cytokines produced by the cells from leaving the cells so that accurate cytokine release rates can be visualized by FACS.

### Example 1

This example demonstrates that the present methods successfully identified tumor antigen-specific T cells and isolated T cell receptors quickly with minimal hands-on culture time.

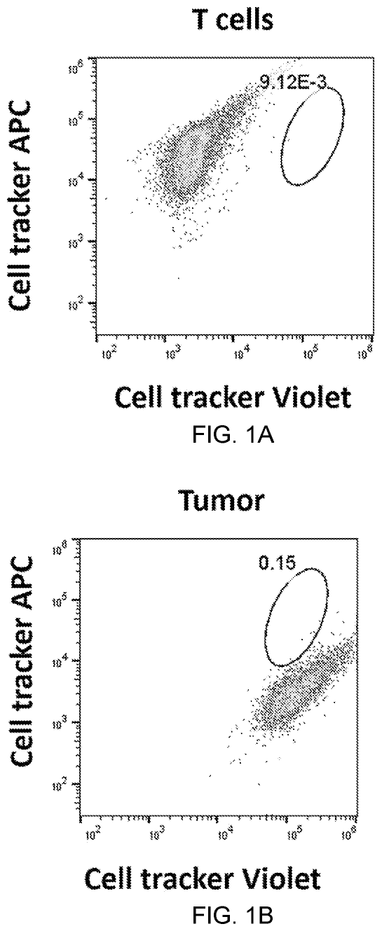

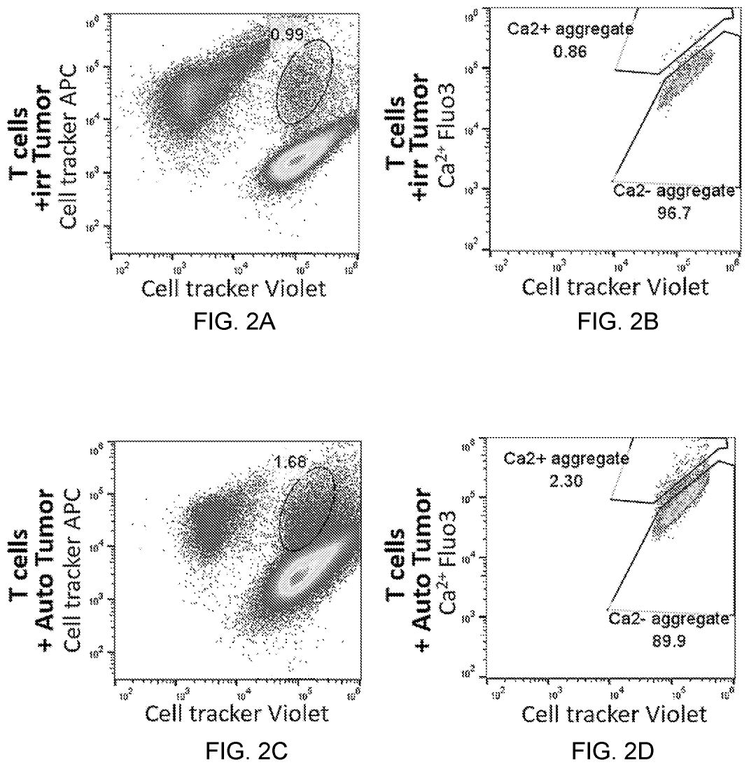

In this study, tumor antigen-specific T cells were identified using an autologous melanoma tumor. Patient 1 had a melanoma tumor and received autologous T cells from Donor 1. Patient 1 and Donor 1 were mismatched for major histocompatibility complex (MHC) class 1. The cells were gated and sorted (FIGS. 1A-2D). Eighteen T cell/target cell aggregates were gated and sorted (FIGS. 2A-2D). Nine TCRa-TCRb pairs were found and 2 were confirmed to be the same as a TCRa-TCRb pair in the autologous tumor. The entire process from start to finish took 7 days or less to complete.

### Example 2

This example further demonstrates that the present methods successfully identified tumor antigen-specific T cells and isolated T cell receptors quickly with minimal hands-on culture time.

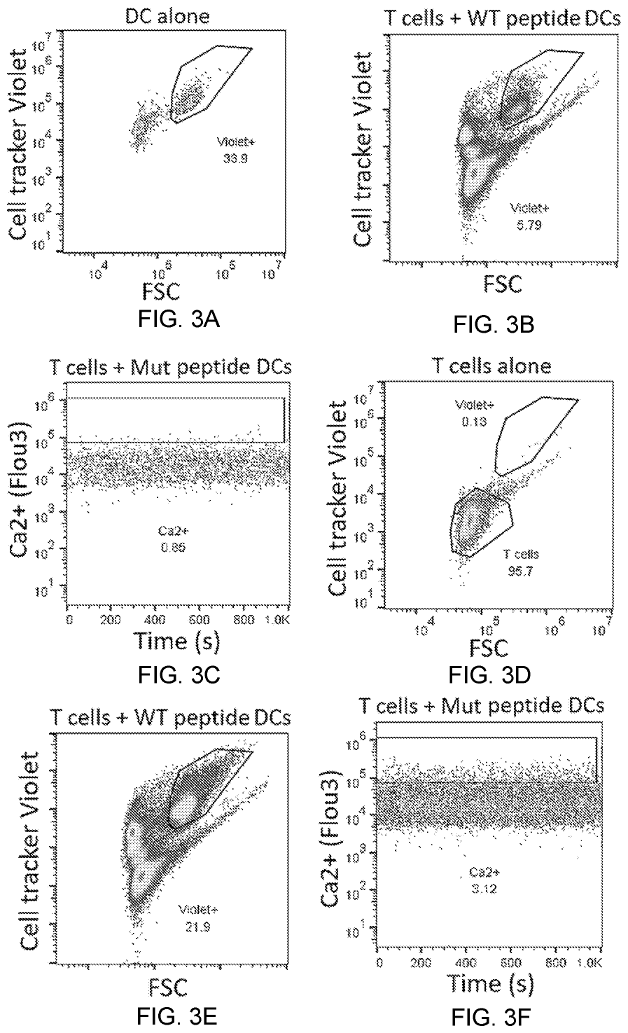

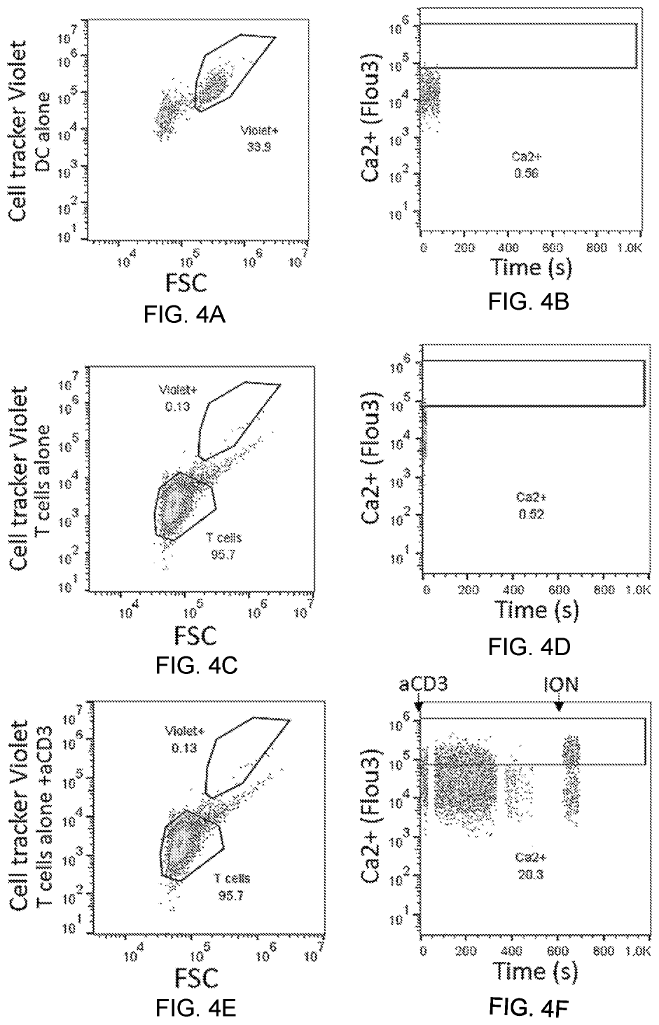

In this study, tumor antigen-specific T cells were identified using an autologous ovarian tumor. Patient 2 had ovarian carcinoma. A tumor fragment was used as the source of T cells. Autologous dendritic cells were pulsed for 2 hours with a 15 mer USP9Xmutant or wild type (1 ug/ml) (target cells from the tumor fragment). The cells were gated and sorted (FIGS. 3A-4F). Eight TCRa-TCRb pairs were found and 2 were confirmed to be the same as a TCRa-TCRb pair specific for USP9Xmutant. The entire process from start to finish took 7 days or less to complete.

### Example 3

This example further demonstrates that the present methods successfully isolated T cell receptors following Ca²⁺ flux with autologous TIL and tumor cells.

In this study, tumor antigen-specific T cells were identified from a patient (3759) with melanoma. Eighteen aggregates were sorted and 8 productive TCRa-TCRb pairs were found, 4 of which were unique receptor pairs. The TCRs were cloned into retroviral vectors and transduced into autologous T cell-enriched PBL. The efficiency of the transduction (how well the mouse TCRb receptors were expressed) was evaluated. The mock (background) was very low at 0.8% and the percentages of expression for 3759-A1, 3759-A3, 3759-A4, and 3759-A12 were 40.0%, 47.6%, 40.4%, and 35.1%, respectively.

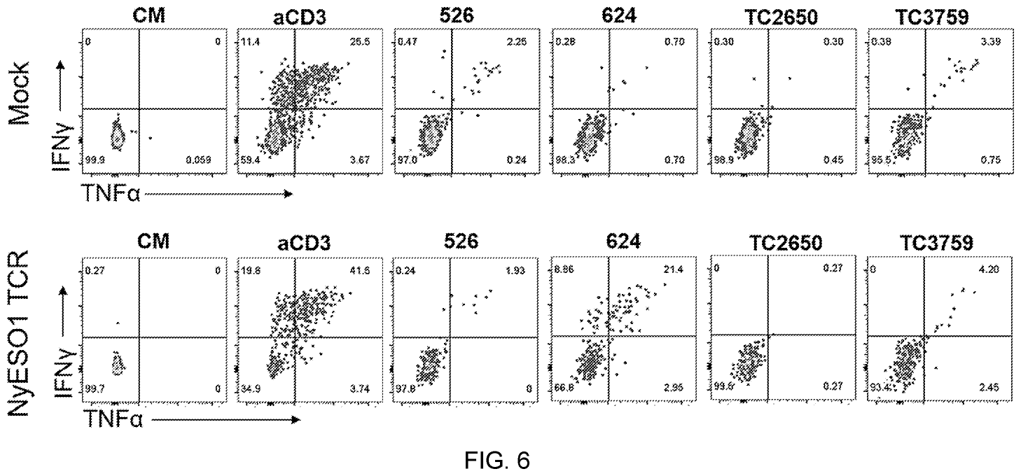

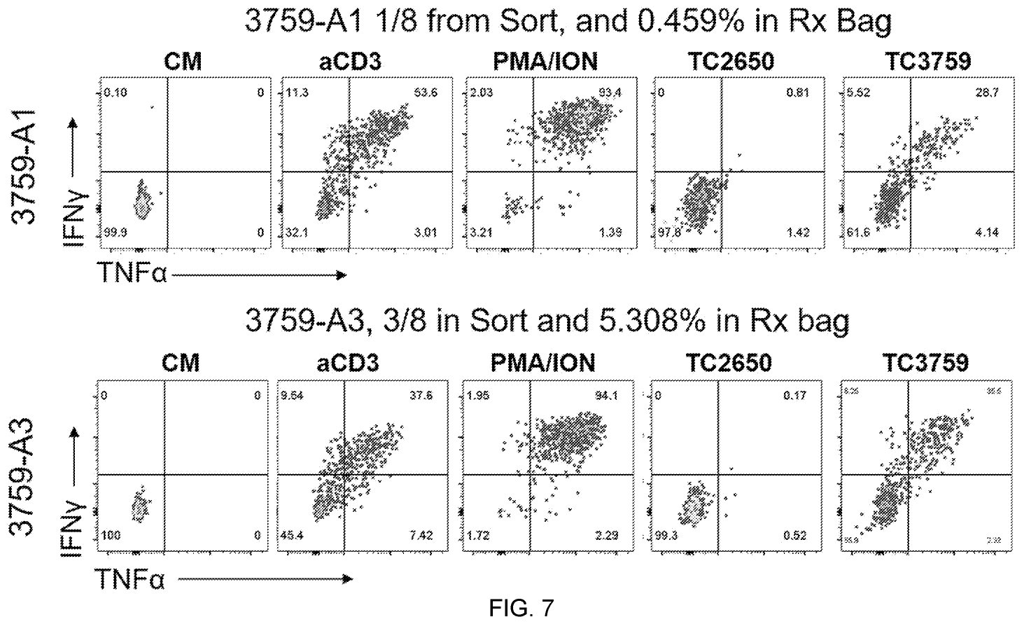

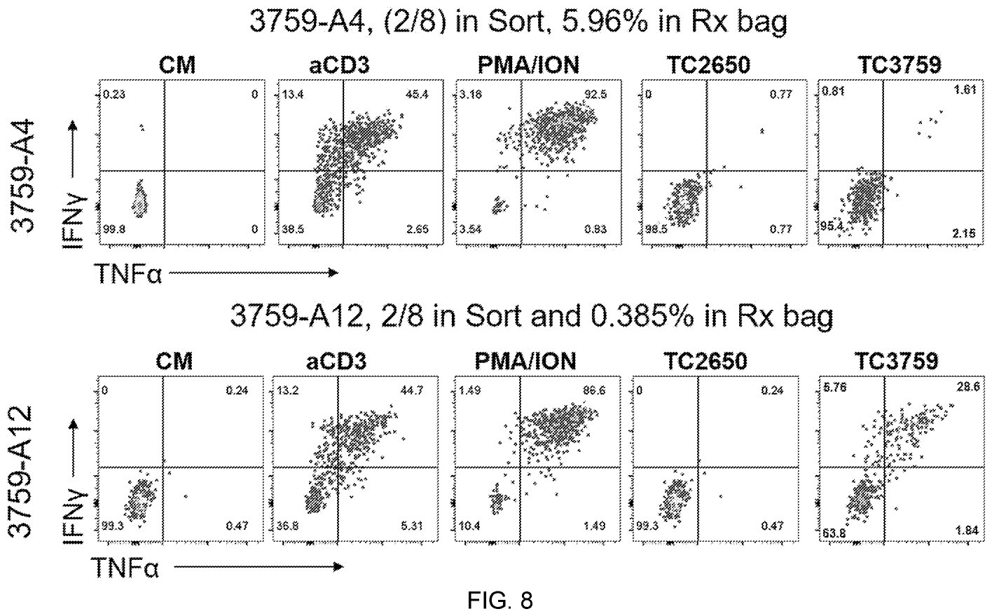

The ability of the TCRs to recognize the tumor was then evaluated. Of the 4 TCR pairs (3759-A1, 3759-A3, 3759-A4, and 3759-A12) that were isolated, 3 conferred specific recognition of the tumor as assessed by specific cytokine release (3759-A1, 3759-A3, and 3759-A12) (FIGS. 6-8).

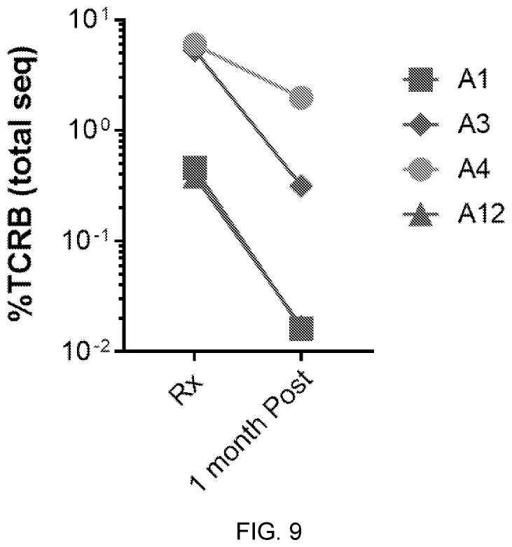

The patient received an infusion containing the 4 TCR pairs. One month post infusion, patient 3759 had the identified TCRa-TCRb pairs present in their blood (FIG. 9).

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of producing an isolated population of cells for adoptive cell therapy, the method comprising:
a) providing a tumor sample containing T cells and tumor cells from a patient having a tumor;
b) separating the T cells from the tumor cells of the tumor sample of a) to produce a separated population of T cells and a separated population of tumor cells;
c) exposing the separated population of T cells of b) to at least one non-cytotoxic cell permeable Ca²⁺ dye to produce dyed T cells;
d) exposing target cells to at least one non-cytotoxic cell membrane dye to produce dyed target cells, wherein the target cells are the separated population of tumor cells of b) or antigen presenting cells (APCs), wherein the separated population of tumor cells of b) express one or more tumor antigens and the APCs are loaded with or express one or more tumor antigens;
e) exposing the dyed T cells to the dyed target cells under conditions sufficient for at least a portion of the dyed T cells to specifically bind to the one or more tumor antigens of the dyed target cells;
f) identifying the dyed T cells which exhibit both (i) specific binding to the dyed target cells and (ii) absorption of a level of the at least one cell permeable Ca²⁺ dye sufficient to indicate T cell receptor activation;
g) separating the dyed T cells identified to exhibit both (i) and (ii) from dyed T cells which fail to exhibit both (i) and (ii);
h) obtaining a sequence of a T cell receptor from a T cell which exhibits both (i) and (ii); and
i) inserting the sequence of the T cell receptor of h) into a peripheral blood mononuclear cell (PBMC) to provide an isolated population of cells for adoptive cell therapy.

2. The method according to claim 1, wherein fluorescence-activated cell sorting (FACS) is used in f) and/or g).

3. The method according to claim 1, wherein i) is completed in less than 7 days after a).

4. The method according to claim 1, wherein the ratio of dyed T cells to dyed target cells in f) is from about 1:5 to about 1:10.

5. The method according to claim 1, wherein the T cell receptor of h) specifically binds to the one or more tumor antigens of the dyed target cells.

6. The method according to claim 1, wherein the PBMC are transduced with a vector comprising the sequence of the T cell receptor of h) to provide the isolated population of T cells for adoptive cell therapy.

7. The method according to claim 6, wherein the vector is a retroviral vector.

8. The method according to claim 1, wherein the PBMC are autologous to the patient.

9. The method according to claim 1, further comprising culturing the PBMC in the presence of interleukin-2 (IL-2), interleukin-7 (IL-7), interleukin-15 (IL-15), interleukin-12 (IL-12), or a combination of two or more of the foregoing.

10. The method according to claim 1, wherein the patient has melanoma.

11. The method according to claim 1, wherein the patient has ovarian cancer.

12. The method according to claim 1, wherein the T cells of a) are tumor infiltrating lymphocytes (TIL).

13. The method of claim 1, wherein the cell membrane dye fluoresces when the dye is bound to the cell membrane of a cell.

14. The method of claim 1, wherein the at least one cell permeable Ca²⁺ dye fluoresces in the presence of Ca²⁺.

15. The method of claim 1, wherein the at least one cell permeable Ca²⁺ dye comprises 4-(6-acetoxymethoxy-2,7-dichloro-3-oxo-9-xanthenyl)-4′-methyl-2,2′(ethylenedioxy)dianiline-N,N,N′,N′-tetraacetic acid tetrakis(acetoxymethyl) ester.

16. The method of claim 1, wherein the at least one cell permeable Ca²⁺ dye comprises glycine, N-[2-[(acetyloxy)methoxy]-2-oxoethyl]-N-[5-[2-[2-[bis[2-[(acetyloxy)methoxy]-2-oxoethyl]amino]-5-methylphenoxy]ethoxy]-2-[(5-oxo-2-thioxo-4-imidazolidinylidene)methyl]-6-benzofuranyl].

17. An isolated population of T cells produced by the method according to claim 1.

18. A pharmaceutical composition comprising the isolated population of cells of claim 17 and a pharmaceutically acceptable carrier.

19. (canceled)

20. A method of treating or preventing cancer in a patient, the method comprising producing an isolated T cell population according to the method of claim 1 and administering the isolated cell population produced by the method to the patient in an amount effective to treat or prevent cancer in the patient.

21. The method according to claim 2, wherein i) is completed in less than 7 days after a).
